(19) United States
(12) Patent Application Publication
(10) Pub. No.: US 2021/0275132 A1
(43) Pub. Date: Sep. 9, 2021

(54) Ultrasonic CT Device

(71) Applicant: Hitachi, Ltd., Tokyo (JP)

(72) Inventors: Kenichi KAWABATA, Tokyo (JP); Kazuhiro YAMANAKA, Tokyo (JP); Atsurou SUZUKI, Tokyo (JP); Yushi TSUBOTA, Tokyo (JP); Takahide TERADA, Tokyo (JP)

(21) Appl. No.: 17/112,004

(22) Filed: Dec. 4, 2020

(30) Foreign Application Priority Data
Mar. 4, 2020 (JP) 2020-037121

(51) Int. Cl. A61B 8/08; A61B 8/00

(52) U.S. Cl. CPC A61B 8/0825; A61B 8/4281; A61B 8/4494; A61B 8/4254; A61B 8/4422; A61B 8/54

(57) ABSTRACT

Provided is an ultrasonic CT device capable of shaping a measurement part such as a breast of a measurement target into a shape suitable for measurement and reducing burden on the measurement target. The ultrasonic CT device includes a tubular measurement container, and a transducer array configured to transmit ultrasonic waves to a measurement target inserted in the measurement container and receive ultrasonic waves from the measurement target. Gel is disposed in the measurement container, and a surface of the gel is inclose contact with at least a surface of the measurement target to which the ultrasonic waves are transmitted. The ultrasonic waves transmitted from the transducer array pass through the gel and are emitted to the measurement target from the surface in close contact with the measurement target.

### TECHNICAL FIELD

The present invention relates to an ultrasonic CT device that processes a signal obtained by emitting ultrasonic waves into a body to generate and display a cross-sectional image of a living body.

### BACKGROUND ART

PTL 1 describes a breast ultrasonic computed tomography (CT) device as a medical diagnosis device in which ultrasonic measurement is applied to breast cancer detection. In the breast ultrasonic CT device, a ring-shaped transducer array which is an ultrasonic transmitter and receiver is disposed around a breast inserted into water, ultrasonic waves are emitted to the breast from 360° in an entire circumferential direction, reflected signals or transmitted signals from the breast are measured, and an image is reconstructed. Accordingly, a tomographic image of the breast is obtained. Information about a structure of abreast tissue is obtained from the reflected signals, and information about a sound speed and attenuation of the ultrasonic waves in the tissue is obtained from the transmitted signals. Generally, a sound speed and an attenuation amount of ultrasonic waves in a tumor are higher than those in normal tissues such as surrounding mammary glands and fat. Therefore, the tumor can be quantitatively detected from a tomographic image (transmitted wave image) of the sound speed or the attenuation amount of the ultrasonic waves.

PTL 2 describes a breast image diagnosis device using a photoacoustic effect. In this device, laser light is emitted in a direction from a nipple to a chest wall, and acoustic signals generated from the breast are measured by a transducer array disposed around the breast to detect a tumor. At this time, the technique of PTL 2 describes a configuration in which the breast is compressed by pushing the breast with a balloon from the nipple to the chest wall to reduce a thickness of the breast. By compressing the breast to reduce the thickness thereof, attenuation of the laser light in the breast can be reduced, and the light can be emitted into all regions of the breast.

PTL 3 proposes a shaping method in which a breast is extended into a cylindrical shape by suctioning a nipple portion of the breast from below and pulling the nipple portion downward in order to reduce an emission angle of ultrasonic waves to a breast surface in a breast ultrasonic CT device.

Further, as shown in Non-patent Literature 1, an ultrasonic CT device is also used for measuring biometric information on targets other than a breast.

### CITATION LIST

### Patent Literature

PTL 1: US Patent Application Publication NO. 2018/0140273

PTL 2: US Patent Application Publication NO. 2016/0262628

PTL 3: US Patent Application Publication NO. 2017/0224305

### Non-Patent Literature

Non-patent Literature: Wiskin, J. et al., SPIE Medical Imaging, issued by SPIE, Volume 10955, MI (2019)

### SUMMARY OF INVENTION

### Technical Problem

In the breast ultrasonic CT device, as described in PTL 1 above, the breast is inserted into a container containing water having a sound speed close to that of the breast tissue, the ultrasonic waves are emitted horizontally (parallel to a main plane of a bed) to the breast from the ring-shaped transducer array through the water around the breast, and reflected waves and transmitted waves thereof are received by the transducer array. However, in general, a shape of the breast is close to a cone, and when the ultrasonic waves are emitted horizontally to the breast, the ultrasonic waves are refracted at a surface of the breast due to a difference between the sound speed of the water filling the breast around and a sound speed of skin of the breast. Since a refraction direction is a direction (z direction) orthogonal to a plane where the transducer array is provided, a proportion of the ultrasonic waves reflected in the breast and the ultrasonic waves transmitted through the breast reaching the transducer array is reduced, which hinders improvement of an image quality.

Further, since the shape of the breast is not a perfect cone and an angle of inclination differs depending on parts, there is a region where the ultrasonic waves are emitted on the surface of the breast with large inclination and a region where the ultrasonic waves are emitted at a close vertical angle, and a distribution of accuracy is generated in the image quality. In particular, there is a problem that at a base of the breast near the chest wall, the surface of the breast has large inclination and a fine image is difficult to be obtained.

Since the breast ultrasonic CT device performs measurement by inserting the breast into the container filled with water, the breast is pushed toward the chest wall due to buoyancy of the water and becomes flat, and the emission angle of the ultrasonic waves on the breast surface increases.

When the breast becomes flat due to the buoyancy of water, the tumor located at the base (near the chest wall) of the breast may be pushed toward a chest wall direction and pushed outside a region (field of view) where the ultrasonic waves can be emitted by the ring-shaped transducer array.

Further, a breast having a small volume is often flat and is easily deformed due to the buoyancy and affected by the emission angle of the ultrasonic waves.

For such reasons, it is desirable to tailor the shape of the breast so that the ultrasonic waves can be emitted to the breast surface as perpendicularly as possible or at a close vertical angle.

The photoacoustic technique of PTL 2 discloses that the breast is compressed by pushing the breast with the balloon from the nipple to the chest wall direction, but a side surface shape of the breast is not considered.

The breast shaping method of PTL 3 is a technique in which a suction device is attached to the nipple portion and the breast is pulled downward to extend into a cylindrical shape. For a patient, it is a psychological burden that the suction device is attached to the nipple portion and the nipple portion is pulled by the device. In addition, it is necessary to add a device or a mechanism for suctioning the breast to a device configuration, which leads to an increase in device cost.

An object of the invention is to provide an ultrasonic CT device capable of shaping a measurement part such as a breast of a measurement target into a shape suitable for measurement and reducing burden on the measurement target.

### Solution to Problem

In order to achieve the above-described object, according to the invention, there is provided an ultrasonic CT device including a tubular measurement container, and a transducer array configured to transmit ultrasonic waves to a measurement target inserted in the measurement container and receive ultrasonic waves from the measurement target. Gel is disposed in the measurement container, and a surface of the gel is in close contact with at least a surface of the measurement target to which the ultrasonic waves are transmitted. The ultrasonic waves transmitted from the transducer array pass through the gel and are emitted to the measurement target from the surface in close contact with the measurement target.

### Advantageous Effect

According to the invention, the measurement part such as the breast of the measurement target can be shaped into a shape suitable for measurement by the gel. Since the gel is soft, burden on the measurement target is small.

### BRIEF DESCRIPTION OF DRAWINGS

FIG. 1 is a block diagram showing an example of an ultrasonic CT device according to a first embodiment of the invention.

FIG. 2 is a cross-sectional view of a measurement container of FIG. 1.

FIG. 3 is a cross-sectional view when a sheet 27 is disposed on a bottom of the measurement container in FIG. 1.

FIGS. 4A to 4C are cross-sectional views showing an operation (first operation example) of each unit during measurement of the ultrasonic CT device according to the first embodiment.

FIG. 5 is a flowchart showing the operation (first operation example) of each unit during the measurement of the ultrasonic CT device according to the first embodiment.

FIGS. 6A to 6C are cross-sectional views showing an operation (second operation example) of each unit during the measurement of the ultrasonic CT device according to the first embodiment.

FIGS. 7A to 7C are cross-sectional views showing an operation (third operation example) of each unit during the measurement of the ultrasonic CT device according to the first embodiment.

FIG. 8 is a flowchart showing the operation (third operation example) of each unit during the measurement of the ultrasonic CT device according to the first embodiment.

FIG. 9 is a block diagram showing an example of an ultrasonic CT device according to a second embodiment of the invention.

FIG. 10 is a block diagram showing an example of an ultrasonic CT device according to a third embodiment of the invention.

FIG. 11 is a block diagram showing an example of an ultrasonic CT device according to a fourth embodiment of the invention.

### DESCRIPTION OF EMBODIMENTS

Hereinafter, an ultrasonic CT device according to an embodiment of the invention will be described with reference to the drawings.

### First Embodiment

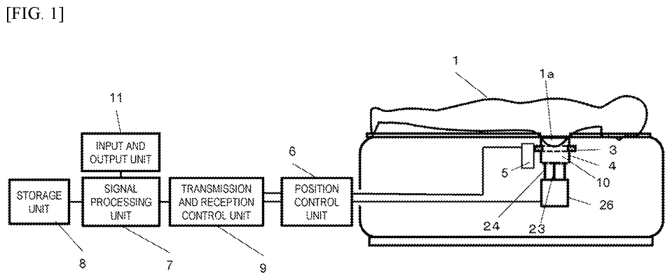

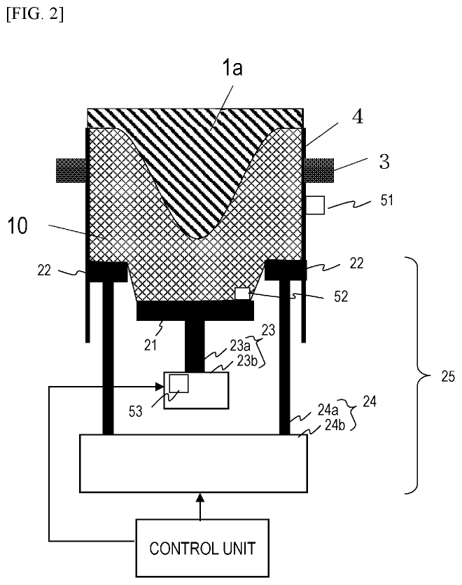

An ultrasonic CT device according to a first embodiment of the invention is a device suitable for breast measurement. FIG. 1 is a block diagram showing an overall configuration of the ultrasonic CT device according to the first embodiment, and FIG. 2 is a cross-sectional view showing a breast at a time of measurement and gel being in close contact with a breast surface.

The breast ultrasonic CT device of the first embodiment includes a bed 2 on which a measurement target 1 is located, a measurement container 4, and a transducer array 3. The bed 2 is provided with an opening into which a measurement part (breast) 1a of the measurement target 1 is inserted. The measurement container 4 has a tubular shape (here, a cylindrical shape), and is disposed under the opening of the bed 2. The transducer array is disposed on an outer circumference of the measurement container 4, transmits ultrasonic waves to the measurement part 1a inserted into the measurement container 4, and receives ultrasonic waves from the measurement part 1a. The transducer array 3 has a ring shape in which a plurality of transducers are arranged along the outer circumference of the measurement container 4 (in a plane parallel to a main plane of the bed 2) and is movable up and down with respect to the measurement container 4.

The transducer array 3 includes a transducer array drive mechanism 5 that moves the transducer array 3 up and down with respect to the measurement container 4. The transducer array drive mechanism 5 is connected to a transducer array position control unit 6 that controls an operation thereof.

Further, the transducer array 3 is connected to a transmission and reception control unit 9 that controls transmission and reception of the ultrasonic waves. The transmission and reception control unit 9 outputs a signal to be transmitted to the transducers forming the transducer array 3 and receives signals received by the transducers. Further, the transmission and reception control unit 9 controls the transducer array position control unit 6 during the measurement to measure a desired cross section of the measurement part 1a (in the plane where the ring-shaped transducer array 3 is disposed).

A signal processing unit 7 is connected to the transmission and reception control unit 9. The signal processing unit 7 generates a reflected wave image and a transmitted wave image of the measurement part la respectively by performing calculation processing, with a predetermined method, on a reflected wave signal and a transmitted wave signal of the ultrasonic waves received by the transducers of the transducer array 3.

An input and output unit 11 and a storage unit 8 are connected to the signal processing unit 7. The input and output unit 11 receives a measurement condition, a calculation condition, and the like from an operator, and displays the generated reflected wave image and the generated transmitted wave image. The storage unit 8 stores the reflected wave signal, the transmitted wave signal, the generated reflected wave image and the generated transmitted wave image.

In the present embodiment, gel 10 is disposed in the measurement container 4 as shown in FIG. 2. A surface of the gel 10 is in close contact with at least a surface of the measurement part 1a to which the ultrasonic waves are transmitted. In the present embodiment, the surface of the gel 10 is in close contact with the entire surface of the measurement part (hereinafter also referred to as the breast) 1a. The ultrasonic waves transmitted from the transducer array 3 passes through the gel 10 and are emitted to the measurement part 1a from the surface (interface) being in close contact with the measurement part 1a. Ultrasonic waves reflected by the measurement part 1a or transmitted through the measurement part 1a pass through the gel 10 again and are received by the transducer array 3.

The surface of the gel 10 is brought into close contact with the surface of the breast 1a to shape the surface of the breast 1a. That is, since a surface shape of the gel 10 is inclose contact with a shape of the breast 1a and the breast 1a is an elastic tissue without bone, the breast 1a is shaped into a surface shape conforming to the surface shape of the gel 10 by shaping in advance the gel 10 into a desired shape. Further, after the gel 10 is brought into close contact with the surface of the breast 1a, the surface shape of the breast 1a being in close contact with the surface of the gel 10 can be deformed by deforming the surface shape of the gel 10. Accordingly, the breast 1a can be shaped so that the ultrasonic waves are uniformly emitted on the surface of the breast 1a at a close vertical angle.

By transmitting the ultrasonic waves from the transducer array 3 to the breast 1a shaped by the gel 10, an angle at which the ultrasonic waves are refracted on the surface of the breast 1a can be reduced, and a proportion of the reflected waves and the transmitted waves in the breast 1a reaching the transducer array 3 can be increased.

Since the gel 10 has high viscosity and elasticity, even when the gel 10 is in close contact with the surface of the measurement part 1a, the measurement target 1 hardly feels burden.

In this way, according to the breast ultrasonic CT device of the present embodiment, since the measurement part (breast) 1a can be shaped into a shape suitable for the measurement and the ultrasonic waves can be transmitted and received, measurement accuracy can be improved. In addition, the burden on the measurement target 1 is light.

The surface shape of the gel 10 may be shaped before the measurement part 1a is inserted into the measurement container 4, or the surface shape of the gel 10 may be deformed into a shape suitable for the measurement after the measurement part 1a is inserted into the measurement container 4 and brought into close contact with the surface of the gel 1a.

### Gel Deformation Mechanism

The ultrasonic CT device of the present embodiment includes a gel deformation mechanism 25 that deforms the gel 10 in the measurement container 4. The gel deformation mechanism 25 deforms the surface shape of the gel 10 by pressing or pulling the gel 10 in the measurement container 4. For example, as shown in FIG. 2, the gel deformation mechanism 25 can use a mechanism disposed on a bottom surface of the measurement container 4 to push a bottom surface of the gel 10 upward and/or pull the gel 10 downward. For example, as shown in FIG. 2, the gel deformation mechanism 25 includes a central plate 21 that is disposed at a central region of the bottom surface of the measurement container 4 and supports a central part of the gel 10, and a peripheral plate 22 that is disposed outside the central plate 21 in a ring shape and supports a peripheral part of the gel 10. The central plate 21 is circular here, but can be any desired shape such as a square. A central drive mechanism 23 having a function of pulling the central plate 21 at least downward is connected to the central plate 21. A peripheral drive mechanism 24 having a function of pushing the peripheral plate 22 at least upward is connected to the peripheral plate 22.

For example, the central drive mechanism 23 includes a shaft member 23a whose upper end is connected to the central plate 21 and a drive source 23b such as a stepping motor that is connected to a lower end of the shaft member 23a and drives the shaft member 23a up and down. Similarly, the peripheral drive mechanism 24 includes a shaft member 24a whose upper end is connected to the peripheral plate 22 and a drive source 24b such as a stepping motor that is connected to a lower end of the shaft member 24a and drives the shaft member 24a up and down. A control unit 26 that controls these operations is connected to the drive sources 23b and 24b. Accordingly, the control unit 26 controls a lowering amount and/or a raising amount of the central plate 21 and the peripheral plate 22, and controls a deformation amount of the gel 10.

As shown in FIG. 2, the gel deformation mechanism 25 having such a configuration can lower a center of the surface of the gel 10 by pulling the central plate 21 downward. Therefore, before the breast 1a is inserted, the gel 10 can be formed with a concave portion to insert the breast 1a by pulling the central plate 21 downward. Further, by pulling the central plate 21 downward after the breast 1a is inserted into the concave portion of the gel 10 and is in close contact with the surface of the gel 10, the gel 10 in close contact with the breast 1a adheres to the breast 1a. Accordingly, a nipple portion of the breast 1a can be pulled to bring the surface of the breast 1a close to an angle perpendicular to the main plane of the bed 2.

Further, by pushing the peripheral plate 22 upward, the gel deformation mechanism 25 can lift the peripheral part of the gel 10 upward, push a base portion (a portion close to the chest wall) of a peripheral edge of the breast 1a, and bring inclination of a surface on the peripheral part of the gel 10 close to the angle perpendicular to the main plane of the bed 2.

Further, the drive sources 23b and 24b may further include a mechanism for rotating the central plate 21 and the peripheral plate 22. Accordingly, since the gel deformation mechanism 25 can change an orientation of the gel 10 by rotating the central plate 21 and/or the peripheral plate 22, the breast 1a and the surface of the gel 10 can be in close contact with each other even when it is difficult to bring them into close contact with each other only by the up-and-down movement.

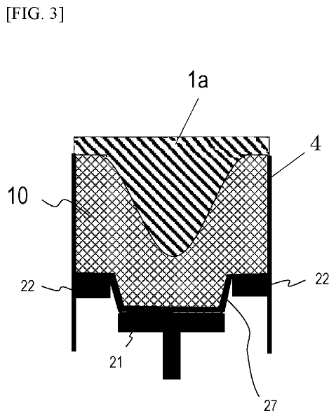

As shown in FIG. 2, when the gel 10 has a self-supporting property, the central plate 21 and the peripheral plate 22 can also serve as the bottom surface of the measurement container. Further, as shown in FIG. 3, a stretchable sheet 27 maybe disposed on the central plate 21 and the peripheral plate 22 to cover the bottom surface of the gel 10. In this case, the sheet 27 can support the gel 10 between the central plate 21 and the peripheral plate 22 even when the gel 10 is soft and has a low self-supporting property.

The measurement container 4 is preferably provided with a sensor that detects whether the surface of the gel 10 and the surface of the breast la are in close contact with each other. Accordingly, the control unit 26 can control the deformation amount of the gel according to a detection result of the sensor. One or more of an acoustic sensor 51, an optical camera 52, and a load sensor 53 may be used as the sensor. As shown in FIG. 2, the acoustic sensor 51 is disposed on a side surface of the measurement container 4, and similarly to the transducer array 3, transmits and receives the ultrasonic waves toward the breast 1a parallel to the main plane of the bed 2. The optical camera 52 may be disposed at any position where the breast 1a can be measured from the gel 10 side. For example, as shown in FIG. 2, the optical camera 52 may be disposed on the bottom surface (central plate 21 or peripheral plate 22) of the measurement container 4, or may be disposed on the side surface. The load sensor 53 measures a load when the drive source 23b moves the plate 21.

When the acoustic sensor 51 is used as the sensor, the ultrasonic waves are transmitted from the acoustic sensor 51 to the breast 1a through the gel 10 and the reflected waves are received by the same acoustic sensor. When a reception signal of the reflected waves is smaller than a preset threshold, the control unit 26 determines that the breast 1a and the gel 10 are not in close contact with each other at the interface and a reception signal of an intensity required for the measurement is not obtained. Further, the ultrasonic waves may be transmitted from the acoustic sensor 51 to the breast 1a through the gel 10, and the transmitted waves transmitted through the breast 1a may be received by another acoustic sensor 51 disposed at a position where the transmitted waves arrive. When the reception signal of the transmitted waves is greater than a preset threshold, the control unit 26 determines that the breast 1a and the gel 10 are in close contact with each other at the interface and the reception signal of the intensity required for the measurement is obtained.

When the acoustic sensor 51 is used as the sensor, the transducer array 3 can also serve as the acoustic sensor 51. Since the transducer array 3 can be driven up and down by the transducer array drive mechanism 5, by disposing the transducer array 3 at any height, it can be confirmed whether contact between the gel 10 and the measurement part 1a at each position is sufficient for the measurement.

Further, when the transducer array 3 also serves as the acoustic sensor 51, an emission angle of the signal of the ultrasonic waves on the surface of the breast 1a can be determined based on a signal intensity of the reflected waves and/or the transmitted waves. That is, when the ultrasonic waves are emitted on the surface of the breast 1a from a direction close to a vertical direction, the intensity of the reflected waves and/or the transmitted waves received by the transducer array 3 increases. Therefore, despite a fact that the surface of the breast 1a and the gel 10 are in close contact with each other, when reflected waves and/or transmitted waves greater than a predetermined threshold cannot be obtained, the control unit 26 may control the surface shape of the gel 10.

On the other hand, when the optical camera 52 is used as the sensor, an image of the breast 1a is scanned through the gel 10, and the control unit 26 determines whether the breast 1a and the gel 10 are in close contact with each other at the interface from the image scanned by the optical camera 52. When the breast 1a and the gel 10 are not in close contact with each other, there is an air layer between the breast 1a and the gel 10. Since the air layer has a large difference in refractive index with respect to the gel 10 and the breast 1a, light is reflected by the air layer and becomes a white region having a high brightness on the scanned image. The control unit 26 can determine whether the breast 1a and the gel 10 are in close contact with each other by determining whether there is the white region having the high brightness by binarizing the image.

When the load sensor 53 is used as the sensor, the load sensor 53 detects a force required to pull the central plate 21 downward. With the breast 1a inserted in the gel 10, the central plate 21 is pulled downward, and when the force required for this is greater than a weight of the gel 10, the control unit 26 determines that the breast 1a is in close contact with the surface of the gel 10.

### First Operation Example of Each Unit during Measurement

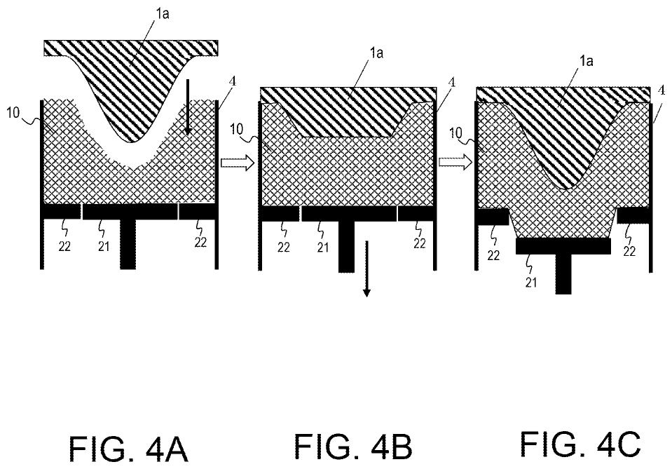

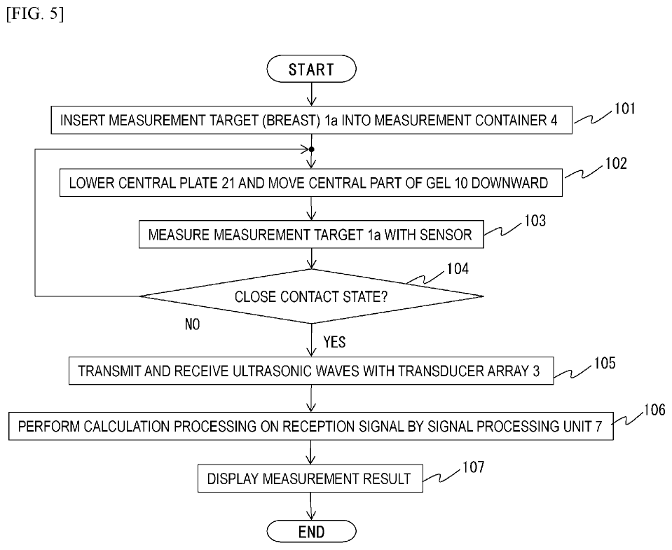

According to the ultrasonic CT device of the present embodiment, an example of a procedure of each unit and an example of an operation of each unit when the breast 1a is measured will be described with reference to FIGS. 4(a) to 4(c) and a flow of FIG. 5.

As shown in FIG. 4, in the container 4, the gel 10 having a concave portion formed on the surface thereof in accordance with the shape of the breast 1a is disposed in advance. In this state, the measurement target 1 lays down on the bed 2 and inserts the breast 1a into the concave portion of the gel 10 of the measurement container 4 (FIG. 4(a), step 101). The breast 1a receives a vertical drag force from the surface of the gel 10 and is pushed upward as shown in FIG. 4(b) to become flat.

Next, the control unit 26 causes the gel deformation mechanism 25 to operate to lower a position of a region of the central part of the bottom surface of the gel 10 to pull downward a central part of the breast 1a contacting an inside of the gel by a predetermined amount (in FIG. 4(c), step 102). Specifically, the control unit 26 causes the drive source 23b to operate to move the central plate 21 downward to pull the central part of the breast 1a downward. Accordingly, while keeping a state where the gel 10 and the breast 1a are in close contact with each other, the shape of the breast 1a can be shaped from a flat shape to a shape close to a natural shape of the breast 1a, or can be extended to a shape that allows the ultrasonic waves to be emitted on the surface of the breast 1a at a close vertical angle.

In a state of FIG. 4(c), in order to adjust the close contact state between the breast 1a and the gel 10 or to finely adjust inclination of the surface of the breast 1a, the drive sources 23b and 24b may further move, under the control of the control unit 26, the central plate 21 and/or the peripheral plate 22 up and down for the fine adjustment (step 102). For example, by raising the peripheral plate 22, the gel 10 can bring the inclination around the base portion of the breast 1a close to inclination perpendicular to the main plane of the bed 2.

Further, in addition to or instead of the up-and-down movement, the central plate 21 and/or the peripheral plate 22 may be rotated.

Next, the control unit 26 measures the close contact state between the breast 1a and the gel 10 with the sensor (step 103). Specifically, for example, the control unit 26 uses the transducer array 3 as a sensor to control the transducer array position control unit 6 and the transmission and reception control unit 9 so that the transducer array 3 is disposed at a predetermined height, the ultrasonic waves are emitted on the breast 1a, and the reflected waves and/or the transmitted waves thereof are received by the transducer array 3. When an intensity of the received signal is equal to or greater than the threshold, the control unit 26 determines that the gel 10 and the breast 1a are in the close contact state (step 104). When the control unit 26 determines that the gel 10 and the breast 1a are not in the close contact state, the process returns to step 102 to adjust the close contact state between the gel 10 and the breast 1a.

In step 104, when the control unit 26 determines that the gel 10 and the breast 1a are in the close contact state, the process proceeds to step 105. The transmission and reception control unit 9 and the transducer array position control unit 6 dispose the transducer array 3 at a predetermined position for the measurement, the ultrasonic waves are emitted on the breast 1a from the transducer array 3 through the gel 10, and the reflected waves or the transmitted waves are received by the transducer array 3 (step 105). The signal processing unit 7 performs the predetermined calculation processing on the reception signal to generate the reflected wave image and/or the transmitted wave image (step 106). The signal processing unit 7 displays an image generated on a display unit of the input and output unit 11 and stores the image in the storage unit 8.

The above process is performed by the control unit 26, the transmission and reception control unit 9, and the transducer array position control unit 6 controlling each unit according to a condition designated by the operator using the input and output unit 11.

### Second Operation Example of Each Unit during Measurement

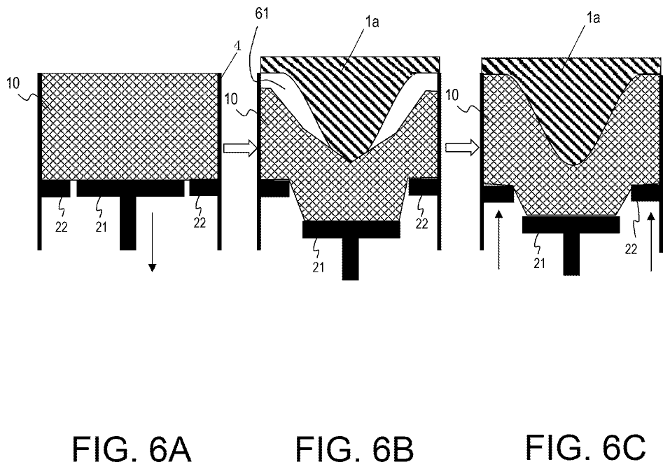

According to the ultrasonic CT device of the present embodiment, another example of a procedure of each unit and another example of an operation of each unit when the breast 1a is measured will be described with reference to FIGS. 6(a) to 6(c).

In the examples of FIGS. 6(a) to 6(c), the gel 10 does not have a concave portion for the breast in advance, and the gel 10 having a flat upper surface is used, which is different from the above-described first operation example of FIGS. 4 (a) to 4 (c) and FIG. 5. Before the measurement, the shape of the gel 10 is deformed to form a concave portion 61 on the surface.

That is, in the second operation example, before step 101 of the first operation example of FIG. 5, as shown in FIG. 6(a), first, by moving the central plate 21 downward, the control unit 26 lowers the central part of the breast 1a and forms the concave portion 61 on the gel surface (see FIG. 6(b)). A distance (movement amount) by which the central plate 21 is moved downward may be a predetermined distance, or when the breast 1a of the measurement target 1 has been measured in the past, the control unit 26 may obtain the movement amount based on measurement data at that time.

Next, steps 101 to 107 of FIG. 5 are performed to insert the breast 1a into the concave portion 61 and shape the breast 1a by the gel deformation mechanism 25, and then the ultrasonic waves are transmitted and received. Since these operations are similar to the flow of first operation example in FIG. 5, description thereof will be omitted.

In this way, in the ultrasonic CT device of the present embodiment, it is not necessary to form a concave portion having a shape corresponding to the breast 1a in advance in the gel 10, and manufacturing cost of the gel 10 can be reduced.

### Third Operation Example of Each Unit during Measurement

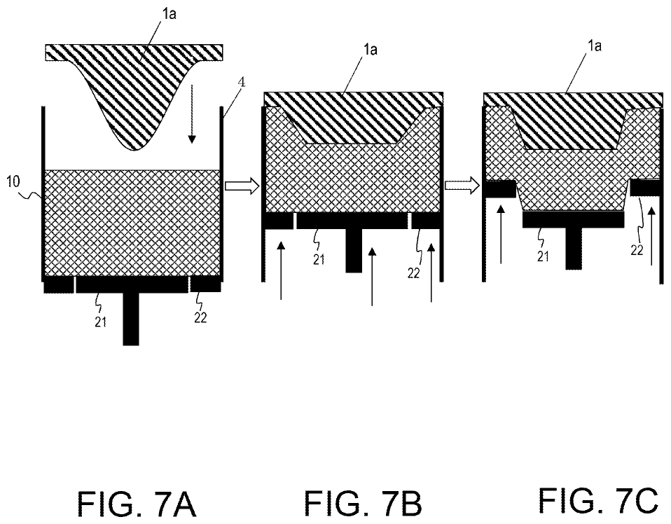

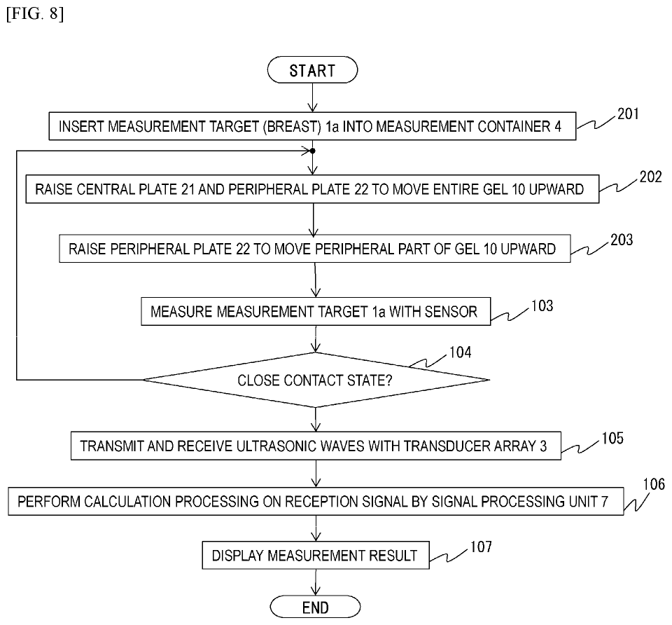

According to the ultrasonic CT device of the present embodiment, another example of a procedure of each unit and an example of an operation of each unit when the breast 1a is measured will be described with reference to FIGS. 7(a) to 7(c) and a flow of FIG. 8.

In the present third operation example, unlike the first operation example and the second operation example described above, without forming a concave portion in advance in the gel 10, as shown in FIGS. 7 (a) to 7 (c), the gel 10 is deformed after the breast 1a is brought into close contact with the gel 10 whose upper surface is flat.

First, as shown in FIG. 7(a), the measurement target 1 inserts the breast 1a into the measurement container 4 (step 201). In this state, by moving both the central plate 21 and the peripheral plate 22 upward, the control unit 26 moves the surface of the gel 10 upward while keeping the surface flat and brings the breast 1a in close contact with the gel 10 due to elasticity of the gel 10 so as to wrap the breast 1a (FIG. 7(b), step 202). At this time, the breast 1a receives a vertical drag force from the surface of the gel 10, and is pressed and becomes flat. Next, the control unit 26 raises the peripheral part of the gel 10 by further raising only the peripheral plate 22 (step 203). Accordingly, the breast 1a receives a force pushing in a central direction from a peripheral edge region of the gel 10, and a side surface of the breast 1a is shaped to be inclined near perpendicularly relative to the main plane of the bed 2 (FIG. 7(c), step 203).

In step 203, after pushing the peripheral plate upward, the control unit 26 may pull the central plate 21 downward. Further, similar to the first operation example, the close contact state between the breast 1a and the gel 10 may be adjusted in the state of FIG. 7(c).

Thereafter, the control unit 26 and the like perform steps 103 to 107 as in the first operation example to measure a degree of close contact, transmit and receive ultrasonic waves, and generate and display an image.

In the present third operation example, since a side surface shape of the breast 1a is shaped to be inclined perpendicularly relative to the main plane of the bed 2, the ultrasonic waves can be emitted on the side surface of the breast 1a from the transducer array 3 at a close vertical angle.

As described above, in the ultrasonic CT device of the first embodiment, since the gel 10 can be brought into close contact with the breast 1a and the ultrasonic waves can be emitted, the breast 1a can be shaped by, with the gel 10, being pulled downward and pushing the peripheral part. Therefore, the proportion of the reflected waves and the transmitted waves of the ultrasonic waves reaching the transducer array 3 can be increased, and the measurement accuracy can be improved.

Moreover, since the gel 10 is elastic and soft, there is an advantage that the measurement target 1 hardly feels a burden even when the measurement part such as the breast 1a is shaped.

### Gel

It is desirable that the gel 10 is capable of satisfying both an acoustic characteristic and a mechanical characteristic necessary for ultrasonic scanning. For example, it is desirable that the gel 10 has a mechanical characteristic, that is, a strain rate when pulled, that is equal to or greater than 100%, preferably equal to or greater than 200%, a sound speed value equivalent to that of water (deviation within 5%) , and an ultrasonic attenuation factor which is equal to or less than 0.1 dB/MHz/cm.

For example, gel obtained by preparing, under a deaeration atmosphere, composite hydrogel of hydrogel polymerized using a radical polymerization initiator and hydrogel by polyvalent ion bond can be used. Specifically, gel that contains polyacrylamide having a mesh structure and alginic acid and in which the alginic acid is retained in a mesh of the mesh structure of polyacrylamide can be obtained. It is desirable that the alginic acid retained in the mesh is crosslinked via an ion to form mesh alginic acid.

When this gel is disposed in the measurement container 4, the gel deforms when the measurement part (breast) la is inserted, and irregularities of the breast 1a can be covered smoothly. Moreover, since the acoustic characteristic of the gel is close to that of water, ultrasonic waves can reach a deep portion for measurement without attenuation.

As a method for manufacturing the above-described gel, first, a plurality of kinds of polymers (hydrogel polymerized using a radical polymerization initiator and hydrogel by polyvalent ion bond or the like) having different polymerization methods or raw materials thereof are mixed. A first kind of polymer (for example, hydrogel polymerized using a radical polymerization initiator) is polymerized or crosslinked to be gelled. Next, a second kind of polymer (for example, hydrogel by polyvalent ion bond) or a raw material thereof is polymerized or crosslinked with the first type polymer to be gelled. By performing all these steps under reduced pressure, the gel capable of satisfying both the acoustic characteristic and the mechanical characteristic necessary for ultrasonic scanning can be manufactured.

The hydrogel generated by polymerization using a radical polymerization initiator is preferably polyacrylamide. The hydrogel generated by crosslinking by polyvalent ion bond is preferably alginic acid crosslinked via a polyvalent ion. As a polyvalent ion source for crosslinking alginic acid, for example, calcium oxalate can be used. A ratio of the hydrogel polymerized via the radical polymerization initiator to the hydrogel generated by crosslinking by polyvalent ion bond can be set to 3:2 to 9:1, and is preferably 13:7 to 9:1.

The present embodiment is not limited to the above-described materials. For example, the hydrogel polymerized using the radical polymerization initiator may include diacetone acrylamide, N-hydroxyethyl acrylamide, or N-(3-methoxypropyl) acrylamide. The hydrogel generated by crosslinking by polyvalent ion bond may include LA gellan gum, carrageenan, and LA pectin.

### Second Embodiment

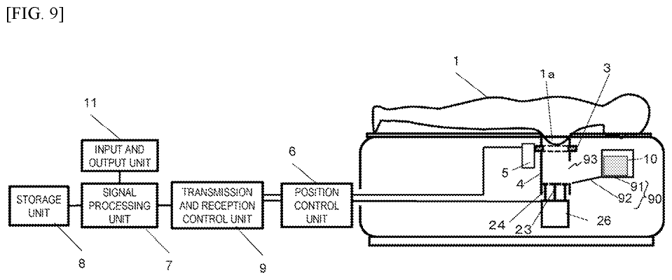

An ultrasonic CT device according to a second embodiment will be described with reference to FIG. 9.

The ultrasonic CT device of the second embodiment includes a gel supply unit 90 that supplies gel into a space inside the measurement container 4. The gel supply unit 90 includes a storage container 91 in which the gel 10 is stored and an introduction path 92 through which the gel 10 in the storage container 91 is introduced into the measurement container 4. The measurement container 4 is provided with an opening 93 through which the gel 10 moved along the introduction path 92 is taken into the internal space of the measurement container 4. The opening 93 may be provided with a door.

The gel 10 is stored in advance in the storage container 91. Before measurement, the gel 10 stored in the storage container 91 is manually moved by an operator or is automatically moved to the measurement container 4. For example, the storage container 91 is provided at a position higher than the opening 93 of the measurement container 4. Further, the introduction path 92 has a slider shape that connects a gel outlet of the storage container 91 and the opening 93 of the measurement container 4. In this case, by manually opening the outlet of the storage container 91 by the operator or automatically opening the outlet, the gel 10 slides along the slider introduction path 92 by its own weight, moves from the opening 93 of the measurement container 4 to the measurement container 4, and is inserted into the measurement container 4.

The storage container 91 may include a heater that keeps the gel 10 warm and a sterilization mechanism that sterilizes (or disinfects) the gel 10. The sterilization mechanism includes, for example, a physical sterilization mechanism such as ultraviolet irradiation or ultrasonic irradiation, or a mechanism for performing chemical sterilization such as reverse soap treatment.

With a configuration of the second embodiment, since the gel 10 can be easily supplied into the measurement container 4, even when the gel 10 is replaced every time the measurement target 1 changes, it does not burden the operator and is hygienic.

Other configurations and operation of each part of the ultrasonic CT device of the second embodiment are the same as those of the first embodiment, and therefore the description thereof will be omitted.

### Third Embodiment

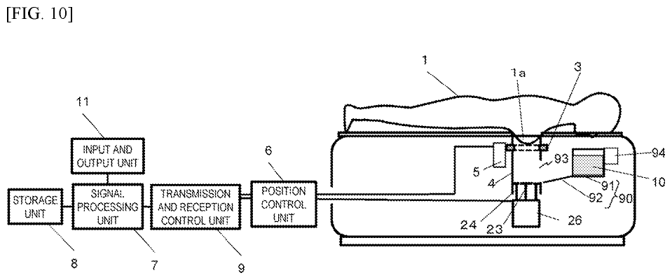

An ultrasonic CT device according to a third embodiment will be described with reference to FIG. 10.

The device of the third embodiment includes the gel supply unit 90 similarly to the device of the second embodiment, but in the gel supply unit 90 of the third embodiment, the storage container 91 also serves as a gel preparation (manufacturing) unit. Specifically, the storage container 91 includes one or more mixing tanks, a raw material supply unit 94 that supplies a raw material to each of the one or more mixing tanks, and a mixing regulator that polymerize or cross-links the raw materials in the mixing tanks to be gelled.

Accordingly, the gel 10 can be prepared (manufactured) by the gel preparation unit (storage container) 91 before measurement, and the gel 10 can be moved to the measurement container 4 manually or automatically during the measurement.

Accordingly, with the device of the third embodiment, as long as raw materials are supplied, the gel 10 can be manufactured and supplied into the measurement container 4, and therefore the operator does not need to prepare the gel 10 and carry it to the storage container 91, which reduces burden on the operator.

Other configurations and operation of each part of the ultrasonic CT device of the third embodiment are the same as those of the first embodiment, and therefore the description thereof will be omitted.

### Fourth Embodiment

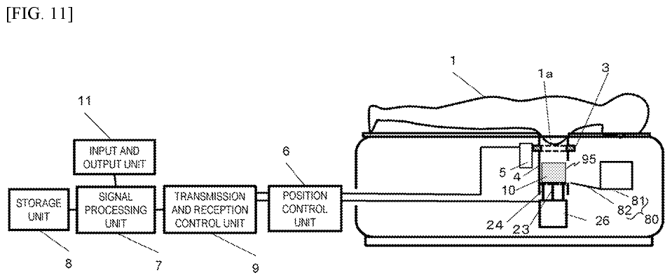

An ultrasonic CT device according to a fourth embodiment will be described with reference to FIG. 11.

The device of the fourth embodiment includes a gel discarding unit 80 in addition to the gel supply unit 90 of the second embodiment or the third embodiment. The gel discarding unit 80 includes a destruction container 81 including a mechanism that performs destruction processing such as crushing on the gel 10, and an introduction path 82 through which the gel 10 in the measurement container 4 is introduced to the destruction container 81. The measurement container 4 is provided with an opening 95 through which the gel in the measurement container 4 is taken out. The opening 95 may also serve as the opening 93 through which the gel of the first embodiment is taken out. The opening 95 may be provided with a door.

As described in the first embodiment, after the breast 1a is shaped and measurement is performed, the gel 10 in the measurement container 4 is moved to the destruction container 81 manually by the operator or is moved automatically. For example, the destruction container 81 is provided at a position lower than the opening 95 of the measurement container 4. Further, the introduction path 82 has a slider shape that connects the opening of the measurement container 4 and a gel intake of the destruction container 81. The door of the opening 95 of the measurement container 4 is manually opened by the operator or is opened automatically. Accordingly, the gel 10 slides along the slider introduction path 92 by its own weight, is taken into the destruction container 81, is subjected to the destruction processing such as crushing in the destruction container 81, and is discharged.

The mechanism that performs the destruction processing of the destruction container 81 is not limited to the crushing, and maybe other processing such as fragmentation with an acid/alkali or thermal dissolution.

Other configurations and operation of each part of the ultrasonic CT device of the fourth embodiment are the same as those of the first embodiment, and therefore the description thereof will be omitted.

### REFERENCE SIGN LIST

1 measurement target
1a measurement part (breast)
2 bed
3 transducer array
4 measurement container
5 transducer array drive mechanism
6 transducer array position control unit
7 signal processing unit
8 storage unit
9 transmission and reception control unit
10 gel
11 input and output unit
21 central plate
22 peripheral plate
23 central drive mechanism
23a shaft member
23b drive source
24 peripheral drive mechanism
24a shaft member
24b drive source
25 gel deformation mechanism
26 control unit
27 sheet
51 acoustic sensor
52 optical camera
53 load sensor
61 concave portion
80 gel discarding unit
81 destruction container
82 introduction path
90 gel supply unit
92 introduction path
93 opening
91 storage container
94 raw material supply unit
95 opening

1. An ultrasonic CT device comprising:
a tubular measurement container; and
a transducer array configured to transmit ultrasonic waves to a measurement target inserted in the measurement container and receive ultrasonic waves from the measurement target, wherein
gel is disposed in the measurement container, and a surface of the gel is in close contact with at least a surface of the measurement target to which the ultrasonic waves are transmitted, and
the ultrasonic waves transmitted from the transducer array pass through the gel and are emitted to the measurement target from the surface in close contact with the measurement target.

2. An ultrasonic CT device comprising:
a tubular measurement container;
a transducer array configured to transmit ultrasonic waves to a measurement target inserted in the measurement container and receive ultrasonic waves from the measurement target; and
a gel supply unit configured to supply gel to a space in the measurement container.

3. The ultrasonic CT device according to claim 2, wherein a surface of the gel in the measurement container is in close contact with a surface of the measurement target, and the ultrasonic waves transmitted from the transducer array are emitted to the measurement target from the surface of the gel.

4. The ultrasonic CT device according to claim 1, wherein the measurement container includes a gel deformation mechanism that deforms a surface shape of the gel by pressing or pulling the gel in the measurement container.

5. The ultrasonic CT device according to claim 2, wherein the measurement container includes a gel deformation mechanism that deforms a surface shape of the gel by pressing or pulling the gel in the measurement container.

6. The ultrasonic CT device according to claim 4, wherein the gel deformation mechanism is disposed on a bottom surface of the measurement container, and pushes a bottom surface of the gel upward or pulls the gel downward.

7. The ultrasonic CT device according to claim 6, wherein the gel deformation mechanism includes a central plate that is disposed at a central region of the bottom surface of the measurement container and supports a central part of the gel, a peripheral plate that is disposed outside the central plate and supports a peripheral part of the gel, a central drive mechanism that pulls the central plate downward, and a peripheral drive mechanism that pushes the peripheral plate upward.

8. The ultrasonic CT device according to claim 7, wherein the central plate and the peripheral plate also serve as the bottom surface of the measurement container.

9. The ultrasonic CT device according to claim 4, further comprising:
a control unit configured to control a deformation amount of the gel by controlling the gel deformation mechanism.

10. The ultrasonic CT device according to claim 9, further comprising:
a sensor configured to detect whether the surface of the gel and the surface of the measurement target are in close contact with each other, wherein
the control unit controls the deformation amount of the gel according to a detection result of the sensor.

11. The ultrasonic CT device according to claim 10, wherein the sensor is a camera, an acoustic sensor, or a load sensor.

12. The ultrasonic CT device according to claim 10, wherein the transducer array also serves as the sensor.

13. The ultrasonic CT device according to claim 2, wherein the gel supply unit includes a storage container in which the gel is stored and an introduction path through which the gel in the storage container is introduced into the measurement container.

14. The ultrasonic CT device according to claim 13, wherein the gel supply unit further includes a gel manufacturing container that manufactures the gel.

15. The ultrasonic CT device according to claim 14, wherein the gel manufacturing container also severs as the storage container.

16. The ultrasonic CT device according to claim 13, wherein the gel supply unit further includes a sterilization mechanism that sterilizes the gel.

17. The ultrasonic CT device according to claim 2, wherein the measurement container further includes a discarding mechanism that takes out and discards the gel in the measurement container.
